(19) United States
(12) Patent Application Publication
(10) Pub. No.: US 2020/0390948 A1
(43) Pub. Date: Dec. 17, 2020

(54) VIBRATION BOX ON NEGATIVE PRESSURE CUP CAPABLE OF STRENGTHENING AIRTIGHTNESS FOR ACHIEVING WATERPROOF, ANTI-COLLISION AND NOISE REDUCTION FUNCTIONS

(71) Applicants: SHIH-TA LIU, Taoyuan City (TW); PO-CHANG LIU, Taoyuan City (TW); BIBOTING International CO., LTD., Taoyuan City (TW)

(72) Inventors: SHIH-TA LIU, Taoyuan City (TW); PO-CHANG LIU, Taoyuan City (TW)

(21) Appl. No.: 16/438,463

(22) Filed: Jun. 12, 2019

(51) Int. Cl. A61M 1/08

(52) U.S. Cl. CPC A61M 1/08; A61M 2205/42; A61M 2205/106

(57) ABSTRACT

A vibration box on a negative pressure cup capable of strengthening airtightness for achieving waterproof, anti-collision and noise reduction functions, comprising a vibration box installed on a negative pressure cup; a vibration motor disposed inside the vibration box; a vibration box back cover disposed on a back of the vibration box for sealing; a waterproof rubber element disposed on the vibration box back cover and a contact surface of the vibration box, the vibration box back cover and the contact surface of the vibration box being reciprocally disposed with flanges and grooves, a surface of the waterproof rubber element being disposed with recesses with a concave cross section, each of the recesses being sleeved to one of the flanges and embedded in the groove to be sealed; a noise reduction cover hooded outside the vibration motor; and a buffer cushion disposed between the vibration motor and the noise reduction cover.

### BACKGROUND OF THE INVENTION

### Field of Invention

The present invention belongs to a vibration box on a negative pressure cup, and more particularly to a vibration box on a negative pressure cup that utilizes a strengthened airtight structure to achieve waterproof, anti-collision and noise reduction functions.

### Related Art

The negative pressure cup is a health care device that covers the surface of the body and stimulates the skin by means of negative pressure suction to achieve the strengthening of body; and in order to strengthen the fitness effect of the negative pressure cup, a vibration box can also be disposed on the negative pressure cup to provide vibration during the negative suction process, for promoting blood circulation and further strengthening the fitness effect.

In order to use it effectively for a long time, the vibration box must avoid moisture from entering the box to cause damage, so it is necessary to strengthen the waterproof function; and, because the vibration box is provided with vibration source by a vibration motor, the vibration motor often collides with the box when it is operating, which not only generates noise, it also reduces the service life and wastes energy.

In terms of waterproof function, the common structure is an O-shaped ring (also known as an O-type ring, an O-ring, an O-type collar), which is a circular-shaped mechanical gasket, usually an elastic body, its cross section is often in circular shape, generally fixed in a recess, and is compressed by two or more components during assembly, thus creating a sealed interface.

Because of its low cost, simple manufacturing, reliable function, simple installation requirements, and the O-shaped ring can withstand pressure of tens of Pascals (thousands of pounds), the O-shaped ring can be used in static applications as well as in dynamic applications where there is relative motion between components, such as the shaft of a rotary pump and the piston of a hydraulic cylinder.

However, due to the simple structure of the O-shaped ring, its fixed contact surface is usually not properly fixed, and also because of changes in the use environment, the O-shaped ring can easily lose its effectiveness in high temperatures or low temperatures, chemical corrosion, or in the process of vibration, wear and movement. Therefore, in addition to the selection of suitable elastomeric materials according to the environmental conditions, the structure needs to be further improved.

Therefore, there is still a need for a solution to solve the above-mentioned problem that the conventional O-shaped rings are susceptible to variations in the assembly method to reduce or lose the waterproof effect.

For the functions of anti-collision and noise reduction, since the working principle of the vibration motor is to adopt the eccentric wheel method, the vibration is caused by the rotation of the eccentric wheel. But the vibration motor is not completely tightly attached to the vibration box, and is usually fixed with a simple frame, and thus the frame can not effectively fix the vibration motor. The vibration motor itself will also sway on the frame during operation, so it is easy to collide with the vibration box. In the long run, it will damage the structure of the vibration box and cause damage to the product.

In addition to damaging the vibration box, arbitrary sway of the vibration motor will cause the vibration of the vibration box to be unstable, resulting in a decrease in performance and affecting the work, which will also result in waste of energy.

Moreover, operation of the vibration motor itself generates vibration noise, plus the sway of the vibration motor and the impact on the vibration box, the noise generated is even more obvious and harsh, seriously affecting the user experience. Especially for health care work as well as in the environment for long-term use, the impact of noise on the user is more significant, and even the willingness to use is reduced, so that the function of the vibration box cannot be exerted.

In view of the aforementioned drawbacks, it is obvious that there is still room for improvement in the conventional technology and it needs to be solved urgently.

With regards to this, the inventor of the present invention set about to improve, through long-term research and development and implementation, a vibration box on a negative pressure cup capable of strengthening airtightness for achieving waterproof, anti-collision and noise reduction functions is proposed.

### SUMMARY OF THE INVENTION

A main object of the present invention is to provide a vibration box on a negative pressure cup capable of strengthening airtightness for achieving waterproof, anti-collision and noise reduction functions, which has good and complete airtightness, and is capable of effectively achieving waterproof, anti-collision and noise reduction functions to meet the needs of users.

Another object of the present invention is to provide a vibration box on a negative pressure cup capable of strengthening airtightness for achieving waterproof, anti-collision and noise reduction functions, which utilizes a waterproof rubber element to solve the problem that the conventional O-shaped rings are susceptible to variations in the assembly method to reduce or lose the waterproof effect, and to obtain the best waterproof effect.

A further object of the present invention is to provide a vibration box on a negative pressure cup capable of strengthening airtightness for achieving waterproof, anti-collision and noise reduction functions, which utilizes a noise reduction cover and a buffer cushion to stabilize the disposition of a closed vibration motor, thereby preventing the conventional vibration motor from colliding with the vibration box to maintain structural integrity and reduce noise.

In order to achieve the above objects, a vibration box on a negative pressure cup capable of strengthening airtightness for achieving waterproof, anti-collision and noise reduction functions, comprising:

a vibration box installed on a negative pressure cup, a vibration motor being disposed inside the vibration box, and a vibration box back cover being disposed on a back of the vibration box for sealing;

a waterproof rubber element disposed on the vibration box back cover and a contact surface of the vibration box, the vibration box back cover and the contact surface of the vibration box being reciprocally disposed with flanges and grooves, and a surface of the waterproof rubber element being disposed with recesses with a concave cross section, and each of the recesses being sleeved to one of the flanges and embedded in the groove to be sealed;

a noise reduction cover hooded outside the vibration motor and fixed on the vibration box to securely fix a position of the vibration motor, and stabilize the vibration motor to reduce impact or noise generated during vibration; and

a buffer cushion disposed between the vibration motor and the noise reduction cover.

Further, the waterproof rubber element is a malleable elastic body, and when the waterproof rubber element is sandwiched between the vibration box back cover and the vibration box, the waterproof rubber element is forcefully expanded to completely fit on the flange and the groove.

In another aspect, the waterproof rubber element is an integrally formed mesh structure formed by a strip surrounding the contact surface.

Alternatively, the waterproof rubber element is a monolithic panel structure that completely covers the back of the vibration box, and the recess is disposed at a position where the panel structure contacts the contact surface.

In addition, the flanges and the grooves of the vibration box back cover and the vibration box are interchangeably disposed, and a direction of the recess of the waterproof rubber element is adjusted to match with the flange and the groove.

Furthermore, the noise reduction cover has a same outer shape as that of an outer casing of the vibration motor to securely fix the noise reduction cover outside the vibration motor.

The buffer cushion is made of foam to effectively ease off the vibration generated by the vibration motor.

Compared with the prior art, the structure of the present invention described above has the following advantages:

1. the waterproof rubber element can achieve the technical efficacies of being relatively more secure, easier to install, not easy to displace, and not easy to fall off in installation, and the waterproof rubber element is not susceptible to variations in the assembly method, otherwise will reduce or lose the waterproof effect;

2. the noise reduction cover is completely hooded outside the vibration motor, and the vibration motor is stably fixed on the vibration box, so that the vibration motor operates more stably without colliding with the vibration box to effectively reduce noise, otherwise the efficiency will be reduced; and

3. the buffer cushion further eases off the sway of the vibration motor itself, making the overall structure more stable and reliable.

### BRIEF DESCRIPTION OF THE DRAWINGS

FIG. 1 is an exploded perspective view of a negative pressure cup and a vibration box according to an embodiment of the present invention;

FIG. 2 is a perspective assembled view of the negative pressure cup and the vibration box according to an embodiment of the present invention;

FIG. 3 is a cross-sectional view of the combination of a vibration box back cover and a waterproof rubber element according to an embodiment of the present invention;

FIG. 3A is an enlarged detail view of the combination portion of the vibration box back cover and the waterproof rubber element of FIG. 3;

FIG. 4 is a cross-sectional view of a waterproof structure combined with the vibration box according to an embodiment of the present invention;

FIG. 4A is an enlarged detail view of the combination portion of the waterproof structure and the vibration box of FIG. 4;

FIG. 5 is a cross-sectional view of the combination of the vibration box, a noise reduction cover and a buffer cushion according to an embodiment of the present invention; and

FIG. 5A is an enlarged detail view of the combination portion of the vibration box, the noise reduction cover and the buffer cushion of FIG. 5.

### DETAILED DESCRIPTION OF THE INVENTION

In order to make the objects, technical solutions and advantages of the present invention more comprehensible, the present invention will be further described in detail below with reference to the accompanying drawings and embodiments. The embodiments and the aspects thereof are described in detail herein, and the description is intended to be illustrative of the structure or the procedure steps of the present invention, which are provided for explanation and not used to limit the scope of claims of the present invention.

An embodiment described in the specification refers to particular features, methods or characteristics described in connection with this embodiment, which are included in at least some embodiments. Therefore, the implementation of various aspects of one embodiment or various embodiments is not necessarily the same embodiment. Furthermore, the features, methods, or characteristics of the present invention can be combined as appropriate in one or more embodiments.

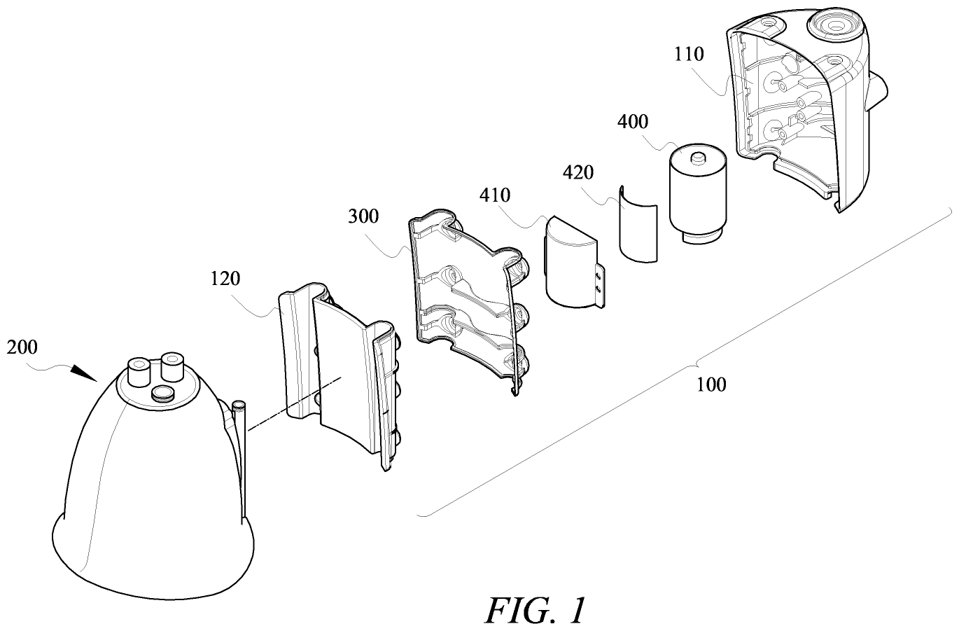

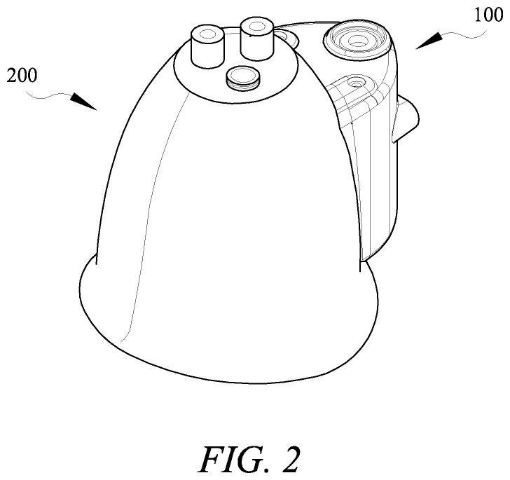

Referring to FIG. 1 and FIG. 2, the present invention provides a vibration box on a negative pressure cup capable of strengthening airtightness for achieving waterproof, anti-collision and noise reduction functions, comprising a negative pressure cup 200 for providing a negative pressure health care function, and a vibration box 100 installed on the negative pressure cup 200 for providing a vibration function.

The vibration box 100 has an accommodation space 110 therein for disposing a vibration motor 400. A back of the vibration box 100 is disposed with a vibration box back cover 120, so that the vibration box back cover 120 covers the back of the vibration box 100 to seal the accommodation space 110.

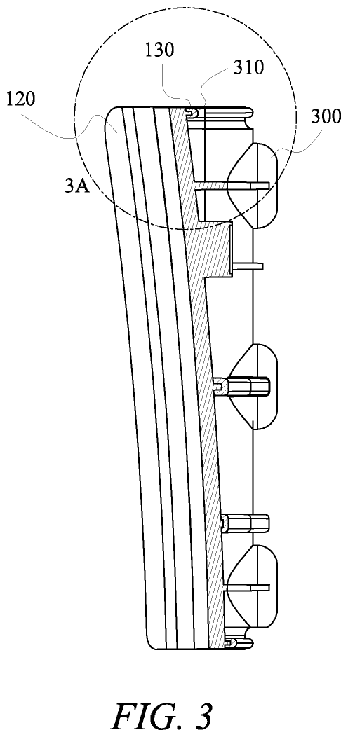

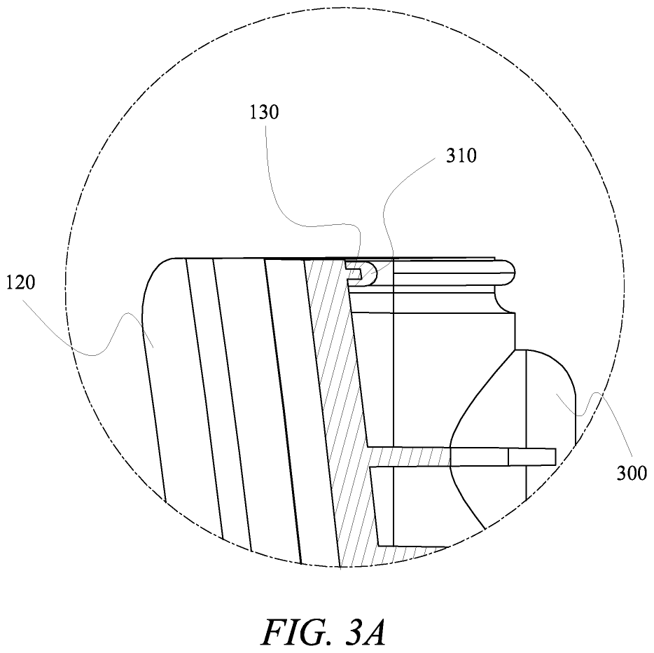

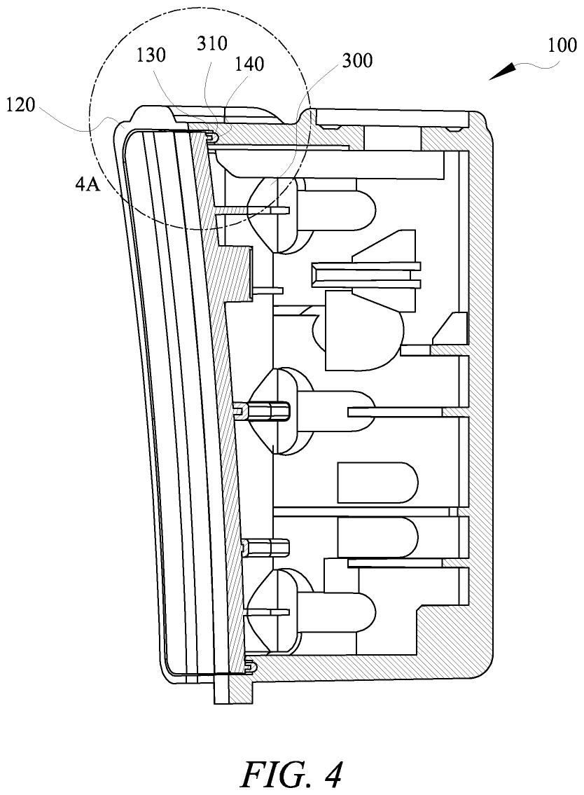

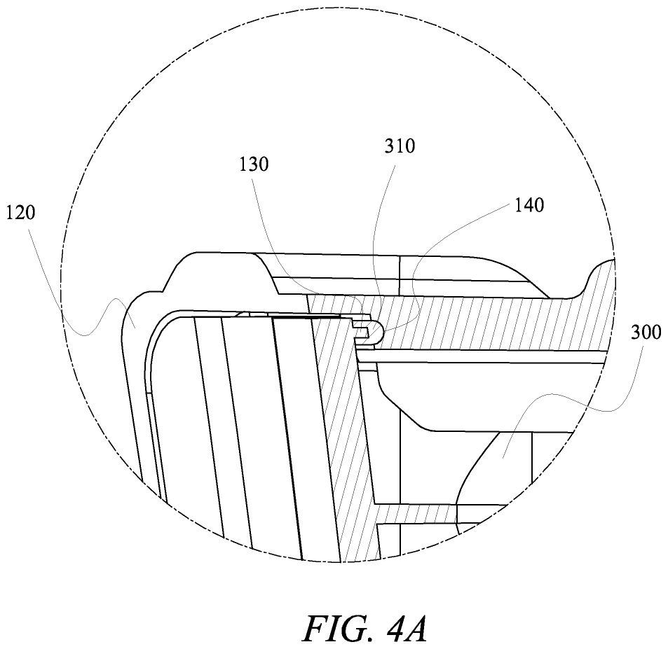

Referring to FIG. 1 to FIG. 4 and FIG. 4A, the present invention mainly provides a waterproof rubber element 300 which is newly applied to prevent water from penetrating into the vibration box 100, and is applied to the waterproof design required for the vibration box 100. In one embodiment, the waterproof rubber element 300 is disposed on the vibration box back cover 120 and a contact surface of the vibration box 100. A surface of the waterproof rubber element 300 is disposed with recesses 310 with a concave cross section, and a shape of the recess 310 is, for example, a U shape or an inverse U shape. In one embodiment, the waterproof rubber element 300 can be molded.

In one embodiment, the waterproof rubber element 300 can be made of synthetic rubbers (also known as thermosetting polymers) such as but not limited to, butadiene rubber (BR), isobutylene isoprene rubber (IIR), chlorosulfonated polyethylene rubber (CSM, complete supplement mixture), ethylene propylene diene monomer (EPDM), ethylene-propylene rubber (EPR), fluororubber (FKM, fluorocarbon), nitrile butadiene rubber (NBR), perfluoroelastomer (FFKM), polyacrylate rubber (ACR), chloroprene rubber (CR), polyisoprene rubber (IR), polysulfide rubber (PSR), silicone rubber (SIR), styrene butadiene rubber (SBR); or thermoplastics such as but not limited to, thermoplastic elastomers (TPE) (e.g. styrene), thermoplastic polyolefin (TPO) (e.g. LDPE low density polyethylene, HDPE high density polyethylene, LLDPE linear low density polyethylene, ULDPE ultra low density polyethylene), thermoplastic polyurethane (TPU) (e.g. polyether, polyester), thermoplastic polyester/polyether elastomers (TEEE), thermoplastic polyamide (TPA) (e.g. polyamide), melt processable rubber (MPR), thermoplastic vulcanizate (TPV).

The U-shaped or inverse U-shaped recess 310 of the waterproof rubber element 300 can be sleeved on a flange 130 of the vibration box back cover 120, so that a size and a shape of the U-shaped or inverse U-shaped recess 310 of the waterproof rubber element 300 need to match with a size and a shape of the flange 130 of the vibration box back cover 120. The waterproof rubber element 300 that is sleeved on the flange 130 can be further disposed in a groove 140 provided on the vibration box 100 and the contact surface of the vibration box back cover 120, so that an outer side surface of the waterproof rubber element 300 is in close contact with the groove 140, and the recess 310 is sleeved on the flange 130 and embedded in the groove 140 to be completely sealed, thereby achieving a waterproof effect. The U-shaped or inverse U-shaped recess 310 of the waterproof rubber element 300 can be sleeved on the flange 130, so that it is easier to install, not easy to displace, and not easy to fall off in installation, and is not susceptible to variations in the assembly method, otherwise will reduce or lose the waterproof effect.

Further, the waterproof rubber element 300 is a malleable elastic body made of the foregoing materials, and when the waterproof rubber element 300 is sandwiched between the vibration box back cover 120 and the vibration box 100, the waterproof rubber element 300 is forcefully expanded to completely fit on the flanges 130 and the grooves 140.

The flange 130 of the vibration box back cover 120 and the groove 140 of the vibration box 100 are designed to be interchangeably disposed. Of course, a direction of the recess 310 of the waterproof rubber element 300 is adjusted to match with positions of the flange 130 and the groove 140.

In one embodiment, the waterproof rubber element 300 is integrally formed by a strip surrounding the vibration box back cover 120 and the contact surface of the vibration box 100, with appearance like a mesh structure. Alternatively, the waterproof rubber element 300 can be disposed as a monolithic panel structure (not shown in the figures) that is capable of completely covering the back of the vibration box 100, and the recess 310 is disposed only at a position where the panel-like waterproof rubber element 300 contacts the contact surface, so that the panel-like waterproof rubber element 300 completely covers and seals the accommodation space 110 of the vibration box 100.

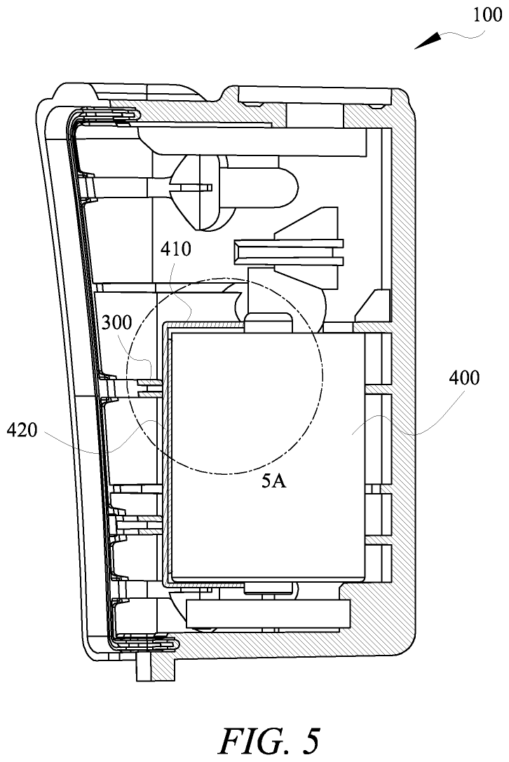

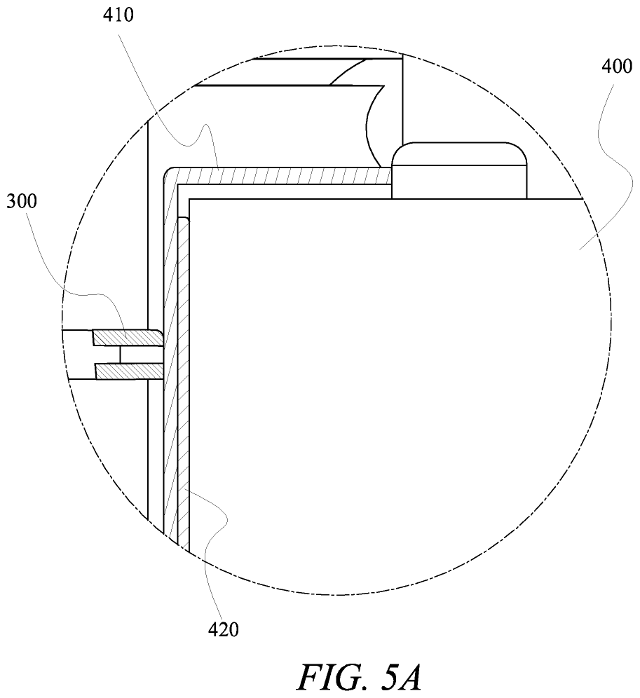

Another focus of the present invention is to provide a noise reduction cover 410 and a buffer cushion 420 for the anti-collision and noise reduction functions of the vibration box 100. Please refer to FIG. 1, FIG. 2, FIG. 5 and FIG. 5A. In one embodiment, the noise reduction cover 410 is hooded outside the vibration motor 400, and the vibration motor 400 is fixed on the vibration box 100 by the noise reduction cover 410 to securely fix a position of the vibration motor 400. Therefore, the vibration motor 400 can be stably operated in the vibration box 100 without arbitrary sway, otherwise will cause the vibration motor 400 to impact on the vibration box 100, and further, due to the cover of the noise reduction cover 410, noise generated by the vibration motor 400 is further reduced.

The noise reduction cover 410 has a same outer shape as that of an outer casing of the vibration motor 400 to securely fix the noise reduction cover 410 outside the vibration motor 400, thereby reducing the gap and effectively preventing noise from being transmitted.

In one embodiment, the buffer cushion 420 is disposed between the vibration motor 400 and the noise reduction cover 410 to ease off the sway of the vibration motor 400 itself.

The material for the soft cushioning of the buffer cushion 420 is mainly foam, but is not limited thereto, so as to surely exert the function of easing off the sway of the vibration motor 400.

In summary, the waterproof rubber element 300 of the present invention has a special shape that matches a size and a shape of the groove 140 of the waterproof structure of the vibration box 100, thereby achieving the technical efficacies of being relatively more secure, easier to install, not easy to displace, and not easy to fall off in installation, and not susceptible to variations in the assembly method, otherwise will reduce or lose the waterproof effect. The noise reduction cover 410 and the buffer cushion 420 further strengthen the fixing of the vibration motor 400, so that it is not easy to sway during operation, reducing the possibility of the vibration motor 400 colliding with the vibration box 100, and maintaining the durability of the product and reducing the occurrence of noise.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A vibration box on a negative pressure cup capable of strengthening airtightness for achieving waterproof, anti-collision and noise reduction functions, comprising a vibration box installed on a negative pressure cup, a vibration motor being disposed inside the vibration box, and a vibration box back cover being disposed on a back of the vibration box for sealing, characterized in that:
a waterproof rubber element being disposed on the vibration box back cover and a contact surface of the vibration box, the vibration box back cover and the contact surface of the vibration box being reciprocally disposed with flanges and grooves, and a surface of the waterproof rubber element being disposed with recesses with a concave cross section, and each of the recesses being sleeved to one of the flanges and embedded in the groove to be sealed;
a noise reduction cover being hooded outside the vibration motor and fixed on the vibration box to securely fix a position of the vibration motor; and
a buffer cushion being disposed between the vibration motor and the noise reduction cover.

2. The vibration box on the negative pressure cup capable of strengthening airtightness for achieving waterproof, anti-collision and noise reduction functions as claimed in claim 1, wherein the waterproof rubber element is a malleable elastic body, and when the waterproof rubber element is sandwiched between the vibration box back cover and the vibration box, the waterproof rubber element is forcefully expanded to completely fit on the flange and the groove.

3. The vibration box on the negative pressure cup capable of strengthening airtightness for achieving waterproof, anti-collision and noise reduction functions as claimed in claim 1, wherein the waterproof rubber element is an integrally formed mesh structure formed by a strip surrounding the contact surface.

4. The vibration box on the negative pressure cup capable of strengthening airtightness for achieving waterproof, anti-collision and noise reduction functions as claimed in claim 1, wherein the waterproof rubber element is a monolithic panel structure that completely covers the back of the vibration box, and the recess is disposed at a position where the panel structure contacts the contact surface.

5. The vibration box on the negative pressure cup capable of strengthening airtightness for achieving waterproof, anti-collision and noise reduction functions as claimed in claim 1, wherein the flange and the groove of the vibration box back cover and the vibration box are interchangeably disposed, and a direction of the recess of the waterproof rubber element is adjusted to match with the flange and the groove.

6. The vibration box on the negative pressure cup capable of strengthening airtightness for achieving waterproof, anti-collision and noise reduction functions as claimed in claim 1, wherein the noise reduction cover has a same outer shape as that of an outer casing of the vibration motor to securely fix the noise reduction cover outside the vibration motor.

7. The vibration box on the negative pressure cup capable of strengthening airtightness for achieving waterproof, anti-collision and noise reduction functions as claimed in claim 1, wherein the buffer cushion is made of foam to effectively ease off the sway of the vibration motor itself.
